(19) United States
(12) Patent Application Publication
(10) Pub. No.: US 2023/0409904 A1
(43) Pub. Date: Dec. 21, 2023

(54) METHOD AND SYSTEM FOR PREDICTING MOLECULAR PROPERTIES

(71) Applicants: Dagnachew Birru, Marlborough (OM); Mukkamala Venkata Sai Prakash, Mumbai (IN); Saisubramaniam Gopalakrishnan, Mumbai (IN); Nareddy Siddartha Reddy, Mumbai (IN); Ganesh Laxman Parab, Mumbai (IN); Vishal Vaddina, Toronto (CA)

(72) Inventors: Dagnachew Birru, Marlborough (OM); Mukkamala Venkata Sai Prakash, Mumbai (IN); Saisubramaniam Gopalakrishnan, Mumbai (IN); Nareddy Siddartha Reddy, Mumbai (IN); Ganesh Laxman Parab, Mumbai (IN); Vishal Vaddina, Toronto (CA)

(73) Assignee: Quantiphi, Inc, Marlborough, MA (US)

(21) Appl. No.: 18/237,516

(22) Filed: Aug. 24, 2023

(51) Int. Cl. G06N 3/08; G06N 5/022

(52) U.S. Cl. CPC G06N 3/08; G06N 5/022

(57) ABSTRACT

A method and system of predicting molecular properties is provided herein. The method includes representing a molecule as a graph and a string. The method further includes encoding the graph into a first feature representation and the string into a second feature representation, using a graph neural network and a transformer-based network, respectively. The method further includes concatenating the first feature representation obtained from the graph neural network and the second feature representation obtained from the transformer-based network to create a combined feature representation. The method further includes fusing the combined feature representation using a linear layer to obtain a synergistic combined feature representation for the molecule. The method further includes predicting one or more molecular properties for the molecule using the synergistic combined feature representation and a predictor network.

### FIELD OF THE INVENTION

The present disclosure relates to molecular properties prediction, and more specifically to a method and system for predicting molecular properties using artificial intelligence techniques.

### BACKGROUND OF THE INVENTION

The prediction of molecular properties plays essential role in diverse scientific and industrial applications, such as drug discovery, material science, and environmental chemistry. Traditional methods for molecular property prediction often rely on experimental techniques, which may be expensive and time-consuming.

One of the fundamental challenges in molecular property prediction is effectively capturing the intricate relationships and characteristics that define molecular structures. Molecular properties, such as solubility, toxicity, and biological activity, are influenced not only by the individual atoms and bonds within a molecule but also by their spatial arrangement and interactions.

In recent years, there has been a growing interest in using machine learning methods to predict molecular properties. Machine learning methods have the potential to be more efficient than traditional methods, and they can be used to predict a wider range of properties. However, existing machine learning methods for predicting molecular properties have some limitations. For example, many existing methods only consider the structural information of a molecule. This may lead to inaccurate predictions, as the chemical information of a molecule also play an important role in determining its properties.

For example, Graph Neural Networks (GNNs) have shown promise in learning representations from graph-structured data, making them a natural choice for capturing the local characteristics of molecules. However, GNNs alone may not adequately capture the broader context and long-range dependencies that are critical for accurately predicting molecular properties.

Therefore, in order to overcome the aforementioned limitations, there exist a need for techniques that may be capable of synergistically fusing the structural and chemical information of the molecule to predict its properties. The proposed techniques use a framework of synergistic fusion that combines pre-trained features from Graph Neural Networks (GNNs) and transformer-based networks, effectively capturing both the local atom-level characteristics and the broader global structure of molecules.

### SUMMARY

The following embodiments presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

Some example embodiments disclosed herein provide computer-implemented method for predicting molecular properties, the method may include representing a molecule as a graph and a string. The method may further include encoding the graph into a first feature representation and the string into a second feature representation, using a graph neural network and a transformer-based network, respectively. The method may further include concatenating the first feature representation obtained from the graph neural network and the second feature representation obtained from the transformer-based network to create a combined feature representation. The method may further include fusing the combined feature representation using a linear layer to obtain a synergistic combined feature representation for the molecule. The method may further include predicting one or more molecular properties for the molecule using the synergistic combined feature representation and a predictor network.

According to some example embodiments, the graph neural network include architectures selected from, but not limited to, a Graph Isomorphism Network (GIN), a Graph Convolutional Network (GCN), and a Graph Attention Networks (GAT), and wherein the transformer based network include architectures selected from, but not limited to, Bidirectional Encoder Representations from Transformers (BERT), Bidirectional Autoregressive Transformers (BART), and Robustly Optimized BERT Pretraining Approach (RoBERTa).

According to some example embodiments, the graph representation comprises nodes representing atoms and edges representing bonds.

According to some example embodiments, the string corresponds to a Simplified Molecular Input Line Entry System (SMILES), a SELF-referencing Embedded Strings (SELFIES), or an International Chemical Identifier (InChi).

According to some example embodiments, the SMILES string, SELFIES string, or InChi are textual representations of the molecule.

According to some example embodiments, predicting the one or more molecular properties comprises estimating solubility, toxicity, reactivity, or biological activity of the molecule.

According to some example embodiments, the predictor network is a neural network that employ one of a regression algorithm or a classification algorithm for predicting molecular properties.

According to some example embodiments, the method further comprising training the predictor network using a loss function that compares the one or more predicted molecular properties with known ground truth properties.

According to some example embodiments, the method further comprising performing back propagation on the predictor network using the loss function to update network parameters; and adjusting weights of the predictor network based on the back propagation to optimize the prediction accuracy of molecular properties.

Some example embodiments disclosed herein provide a computer system for predicting molecular properties, the computer system comprising one or more computer processors, one or more computer readable memories, one or more computer readable storage devices, and program instructions stored on the one or more computer readable storage devices for execution by the one or more computer processors via the one or more computer readable memories, the program instructions comprising representing a molecule as a graph and a string. The one or more processors are further configured for encoding the graph into a first feature representation and the string into a second feature representation, using a graph neural network and a transformer-based network, respectively. The one or more processors are further configured for concatenating the first feature representation obtained from the graph neural network and the second feature representation obtained from the transformer-based network to create a combined feature representation. The one or more processors are further configured for fusing the combined feature representation using a linear layer to obtain a synergistic combined feature representation for the molecule. The one or more processors are further configured for predicting one or more molecular properties for the molecule using the synergistic combined feature representation and a predictor network.

Some example embodiments disclosed herein provide a non-transitory computer readable medium having stored thereon computer executable instruction which when executed by one or more processors, cause the one or more processors to carry out operations for predicting molecular properties, the operations comprising representing a molecule as a graph and a string. The operations further comprising encoding the graph into a first feature representation and the string into a second feature representation, using a graph neural network and a transformer-based network, respectively. The operations further comprising concatenating the first feature representation obtained from the graph neural network and the second feature representation obtained from the transformer-based network to create a combined feature representation. The operations further comprising fusing the combined feature representation using a linear layer to obtain a synergistic combined feature representation for the molecule. The operations further comprising predicting one or more molecular properties for the molecule using the synergistic combined feature representation and a predictor network.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The above and still further example embodiments of the present invention will become apparent upon consideration of the following detailed description of embodiments thereof, especially when taken in conjunction with the accompanying drawings, and wherein:

FIG. 1 is a block diagram of an environment of a system for predicting molecular properties, in accordance with an example embodiment.

FIG. 2 is a block diagram illustrating various modules within an Artificial Intelligence (AI) model of a prediction device configured for predicting molecular properties, in accordance with an example embodiment.

FIG. 3 illustrates a functional block diagram for predicting molecular properties, in accordance with an example embodiment.

FIG. 4 illustrates a flow diagram of a method for predicting molecular properties, in accordance with an example embodiment.

FIG. 5 illustrates another flow diagram of a method for predicting molecular properties, in accordance with an example embodiment.

FIG. 6 illustrates a flow diagram of a method for training a predictor network to predict molecular properties, in accordance with an example embodiment.

FIG. 7 shows an exemplary table depicting comparison of a performance of different models for predicting a toxicity of molecules, in accordance with an example embodiment.

FIG. 8 shows an exemplary table depicting a comparison of performance of different models for predicting a solubility of molecules, in accordance with an example embodiment.

FIG. 9 shows an exemplary graphical representation 900 depicting comparison of a performance of different models for predicting a solubility and toxicity of molecules, in accordance with an example embodiment.

FIG. 10 shows a comparison of latent space representations of SYN-FUSION, MolCLR, and MegaMolBART, in accordance with an example embodiment.

FIG. 11 illustrates exemplary Activation Maps of different models, in accordance with an example embodiment.

FIG. 12 illustrates exemplary weight histograms 1200 of different models, in accordance with an example embodiment.

FIG. 13 illustrates exemplary loss interpolation plots of different models, in accordance with an example embodiment.

The figures illustrate embodiments of the invention for purposes of illustration only. One skilled in the art will readily recognize from the following description that alternative embodiments of the structures and methods illustrated herein may be employed without departing from the principles of the invention described herein.

### DETAILED DESCRIPTION

In the following description, for purposes of explanation, numerous specific details are set forth in order to provide a thorough understanding of the present invention. It will be apparent, however, to one skilled in the art that the present invention can be practiced without these specific details. In other instances, systems, apparatuses, and methods are shown in block diagram form only in order to avoid obscuring the present invention.

Reference in this specification to “one embodiment” or “an embodiment” or “example embodiment” means that a particular feature, structure, or characteristic described in connection with the embodiment is included in at least one embodiment of the present invention. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

Some embodiments of the present disclosure will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout.

The terms “comprise”, “comprising”, “includes”, or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., are non-transitory. Examples include random access memory (RAM), read-only memory (ROM), volatile memory, non-volatile memory, hard drives, CD ROMs, DVDs, flash drives, disks, and any other known physical storage media.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present invention. Further, it is to be understood that the phraseology and terminology employed herein are for the purpose of the description and should not be regarded as limiting. Any heading utilized within this description is for convenience only and has no legal or limiting effect.

### Definitions

The term “SMILES” may refer to a Simplified Molecular Input Line Entry System, which is a widely used notation in computational chemistry for representing molecular structures as text strings. SMILES strings provide a concise and standardized way to encode molecular information, including atom types, bond connectivity, and stereochemistry. This notation facilitates the exchange and manipulation of molecular data in various computational and research contexts.

The term “module” used herein may refer to a hardware processor including a Central Processing Unit (CPU), an Application-Specific Integrated Circuit (ASIC), an Application-Specific Instruction-Set Processor (ASIP), a Graphics Processing Unit (GPU), a Physics Processing Unit (PPU), a Digital Signal Processor (DSP), a Field Programmable Gate Array (FPGA), a Programmable Logic Device (PLD), a Controller, a Microcontroller unit, a Processor, a Microprocessor, an ARM, or the like, or any combination thereof.

The term “machine learning model” may be used to refer to a computational or statistical or mathematical model that is trained on classical ML modelling techniques with or without classical image processing. The “machine learning model” is trained over a set of data and using an algorithm that it may be used to learn from the dataset.

The term “artificial intelligence” may be used to refer to a model built using simple or complex Neural Networks using deep learning techniques and computer vision algorithms. Artificial intelligence model learns from the data and applies that learning to achieve specific pre-defined objectives.

### End of Definitions

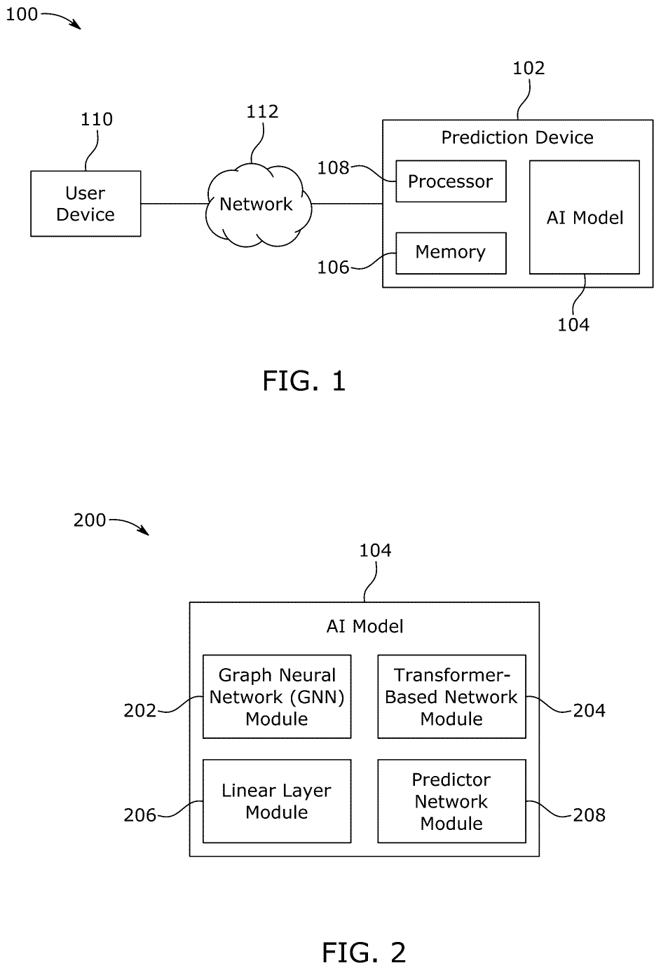

Embodiments of the present disclosure may provide a method, a system, and a computer program product for predicting molecular properties. The method, the system, and the computer program product predicting molecular properties in such an improved manner are described with reference to FIG. 1 to FIG. 12 as detailed below.

FIG. 1 illustrates a block diagram of an environment of a system 100 for predicting molecular properties. The system 100 includes a prediction device 102 and a user device 110 associated with a user. The prediction device 102 may be communicatively coupled with the user device 110 via a network 112. Examples of the prediction device 102 may include, but are not limited to, a server, a desktop, a laptop, a notebook, a tablet, a smartphone, a mobile phone, an application server, or the like.

The network 112 may be wired, wireless, or any combination of wired and wireless communication networks, such as cellular, Wi-Fi, internet, local area networks, or the like. In one embodiment, the network 112 may include one or more networks such as a data network, a wireless network, a telephony network, or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (Wi-Fi), wireless LAN (WLAN), Bluetooth®, Internet Protocol (IP) data casting, satellite, mobile ad-hoc network (MANET), and the like, or any combination thereof.

The prediction device 102 may be capable of predicting one or more molecular properties. By way of an example, the network 110 may facilitate the prediction device 102 to communicate with the user of the user device 110 for predicting the one or more molecular properties. The user device 110 may be a desktop computer, a workstation, a laptop computer, a tablet computer, a personal computer (PC), a personal digital assistant (PDA), a smart phone, or any programmable electronic device capable of communicating via the network 112 with various component and devices within the system 100.

To elaborate, the user may enter molecular formula, (for example, InChi representation, or other molecule identifiers) through a GUI of their user device (such as, a smartphone). The prediction device 102 may then use a framework of synergistic fusion to predict the properties of the molecule. In some embodiments, the results of the prediction may be displayed on the user device 102.

The prediction device 102 may include an Artificial Intelligence (AI) model 104, which may further include various modules that enable the prediction management device 102 to predicting molecular properties. These modules are explained in detail in conjunction with FIG. 2. In order to predict the molecular properties, the prediction device 102 may be configured to representing a molecule as a graph and a string. The graph may include nodes representing atoms and edges representing bonds. The string may correspond to a Simplified Molecular Input Line Entry System (SMILES) or a SELF-referencIng Embedded Strings (SELFIES). The SMILES string and the SELFIES string are textual representations of the molecule.

The prediction device 102 may further encode the graph into a first feature representation and the string into a second feature representation, using a graph neural network and a transformer-based network, respectively.

Further, the prediction device 102 may concatenate the first feature representation obtained from the graph neural network and the second feature representation obtained from the transformer-based network to create a combined feature representation. Further, the prediction device 102 may fusing the combined feature representation using a linear layer to obtain a synergistic combined feature representation for the molecule.

Upon synergistically fusing, the prediction device 102 may predict one or more molecular properties for the molecule using the synergistic combined feature representation and a predictor network. The complete process followed by the system 100 is further explained in detail in conjunction with FIG. 2 to FIG. 10.

The prediction device 102 may further include a memory 106, and a processor 108. The term “memory” used herein may refer to any computer-readable storage medium, for example, volatile memory, random access memory (RAM), non-volatile memory, read only memory (ROM), or flash memory. The memory 106 may include a Random-Access Memory (RAM), a Read-Only Memory (ROM), a Complementary Metal Oxide Semiconductor Memory (CMOS), a magnetic surface memory, a Hard Disk Drive (HDD), a floppy disk, a magnetic tape, a disc (CD-ROM, DVD-ROM, etc.), a USB Flash Drive (UFD), or the like, or any combination thereof.

The term “processor” used herein may refer to a hardware processor including a Central Processing Unit (CPU), an Application-Specific Integrated Circuit (ASIC), an Application-Specific Instruction-Set Processor (ASIP), a Graphics Processing Unit (GPU), a Physics Processing Unit (PPU), a Digital Signal Processor (DSP), a Field Programmable Gate Array (FPGA), a Programmable Logic Device (PLD), a Controller, a Microcontroller unit, a Processor, a Microprocessor, an ARM, or the like, or any combination thereof.

The processor 108 may retrieve computer program code instructions that may be stored in the memory 106 for execution of the computer program code instructions. The processor 208 may be embodied in a number of different ways. For example, the processor 108 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor 108 may include one or more processing cores configured to perform independently. A multi-core processor may enable multiprocessing within a single physical package. Additionally, or alternatively, the processor 108 may include one or more processors configured in tandem via the bus to enable independent execution of instructions, pipelining and/or multithreading.

Additionally, or alternatively, the processor 108 may include one or more processors capable of processing large volumes of workloads and operations to provide support for big data analysis. In an example embodiment, the processor 108 may be in communication with a memory 106 via a bus for passing information among components of the system 100.

The memory 106 may be non-transitory and may include, for example, one or more volatile and/or non-volatile memories. In other words, for example, the memory 106 may be an electronic storage device (for example, a computer readable storage medium) comprising gates configured to store data (for example, bits) that may be retrievable by a machine (for example, a computing device like the processor 108). The memory 106 may be configured to store information, data, contents, applications, instructions, or the like, for enabling the apparatus to carry out various functions in accordance with an example embodiment of the present disclosure. For example, the memory 106 may be configured to buffer input data for processing by the processor 108. The memory 106 may also store various data (e.g., molecular structures, SMILES strings, training datasets, model parameters, prediction results, user preferences, historical predictions, etc.) that may be captured, processed, and/or required by the processor 108 of the prediction device 102 predict one or more molecular properties. This stored data enables the prediction device 102 to access and manipulate essential information during the prediction process, facilitating accurate and informed molecular property predictions based on the synergistic fusion approach.

The user device 110 may include a display. The display may include a user interface. Therefore, the display and the user interface may be used to interact the prediction device 102 with the user device and vice versa for predicting molecular properties based on actions performed by the user device 102.

By way of an example, the display offers a means for presenting information and interacting with users. The user interface (e.g., a graphical platform) enables users to engage with the prediction device 102 and its functionalities. The user interface provides a dynamic and intuitive interaction medium, allowing users to initiate and manage molecular property predictions seamlessly. Through the user interface, users may conveniently input molecular data, such as images or Textual descriptions like SMILES, SELFIES, etc. strings, initiate prediction processes, and visualize the results. The GUI may offer interactive visual representations of molecular structures, enhancing the user's experience and facilitating efficient utilization of the prediction device 102.

Furthermore, the user interface may enable users to customize prediction parameters, select specific molecular properties of interest, and tailor prediction preferences according to their needs. The user interface's responsiveness and visual feedback ensure that users may engage in an informative prediction experience.

FIG. 2 illustrates a block diagram 200 of various modules within the AI model 104 of the prediction device 102 that is configured to predict molecular properties, in accordance with an example embodiment. The AI model 104 may include a Graph neural network (GNN) module 202, a transformer-based network module 204, a linear layer module 206, and a predictor network module 208.

In order to predict the one or more molecular properties (such as, solubility, toxicity, reactivity, or biological activity of the molecule), initially the molecular structure is transformed into two primary representations: a structure-based representation and a text-based representation.

In particular, initially the molecule may be converted into a graph representation and a SMILES string representation. Subsequently, these representations may be fed into the GNN module 202 (for example, a graph neural network) and the transformer-based network module 204 (for example, a transformer-based network) to convert them to their respective feature representations. It should be noted that graphs are a type of data structure that represents relationships between entities, whereas SMILES strings are a type of text representation of molecules.

The graph neural network (GNN) is a type of neural network that is designed to process and analyze graph-structured data. Graph-structured data comprises of nodes and edges, where nodes represent entities (such as atoms in molecules or users in a social network), and edges represent relationships or connections between these entities. GNNs are particularly well-suited for tasks that involve understanding and learning from the complex relationships and dependencies present in graph data.

Here are some examples of GNN:

Graph convolutional networks (GCNs): GCNs are a type of GNN that is used to learn the relationships between nodes in a graph. GCNs are able to learn the local and global structure of a graph, and they can be used to predict a variety of properties, such as the stability and reactivity of a molecule.

Graph attention networks (GATs): GATs are a type of GNN that is used to learn the attention weights between nodes in a graph. GATs are able to learn the importance of different nodes in a graph, and they can be used to predict a variety of properties, such as the solubility and toxicity of a molecule.

Graph recurrent networks (GRNs): GRNs are a type of GNN that is used to learn the temporal relationships between nodes in a graph. GRNs are able to learn the evolution of a graph over time, and they can be used to predict a variety of properties, such as the spread of a disease or the evolution of a social network.

Graph autoencoders (GAEs): GAEs are a type of GNN that is used to learn the latent representation of a graph. GAEs are able to learn the underlying structure of a graph, and they can be used to reconstruct the graph or to predict properties of the graph.

Graph Isomorphism Networks (GIN): Within the realm of GNNs, a specific type known as the GIN has gained attention for its versatility and effectiveness in learning graph representations. The GIN architecture is designed to address the challenge of graph isomorphism, which involves determining whether two graphs are structurally identical (isomorphic).

The key characteristic of GIN is its ability to aggregate information from neighbouring nodes in an iterative and expressive manner. It achieves this by applying neighbourhood aggregation steps followed by non-linear transformations. Specifically, the GIN model updates the features of a node by aggregating information from its neighbours, much like the propagation of information in a graph. This aggregation process is repeated for multiple iterations, allowing the model to capture complex relationships and dependencies within the graph.

One notable aspect of GIN is its permutation-invariant property. This means that GIN is designed to yield the same output regardless of the order in which nodes are processed, making it well-suited for applications where the arrangement of nodes should not impact the model's prediction.

The feature representation obtained from the GNN module 202, such as GIN, encodes the structural and relational information of the molecule's graph representation. It captures details about individual atoms (nodes) and their connections (edges) in the molecular graph. This feature representation may cover local interactions, atom properties, and bond relationships within the molecule.

Apart from GNN, the transformer-based network in the transformer-based network module 204 may represent the chemical information of a molecule as a text. Some examples of transformer-based networks that could be used include:

Bart: BART is a transformer-based network that is developed by Google AI. BART is a powerful model that has been shown to be effective in a variety of tasks, including machine translation, text summarization, and question answering.

T5: T5 is a transformer-based network that is developed by Google AI. T5 is a powerful model that has been shown to be effective in a variety of tasks, including natural language inference, question answering, and summarization.

BERT: BERT, which stands for Bidirectional Encoder Representations from Transformers, is a renowned transformer-based network designed for various natural language processing tasks. BERT has been adapted to the realm of molecular property prediction, where its remarkable capacity to capture intricate relationships and contextual dependencies has proven exceptionally valuable. This model excels in understanding molecular structures and their properties by considering bidirectional relationships within the input data. BERT's proficiency has been demonstrated across a spectrum of properties, encompassing solubility, reactivity, stability, and toxicity, making it a versatile choice for predictive modeling in the field.

RoBERTa: RoBERTa, an extension of the BERT architecture, brings further advancements to transformer-based networks. The name RoBERTa stands for A Robustly Optimized BERT Pretraining Approach,” and it is tailored to refine language understanding tasks. In the context of molecular property prediction, RoBERTa's enhanced training methodology yields representations that encapsulate nuanced features within molecular structures. By utilizing large-scale pretraining, RoBERTa enhances its ability to capture context and semantics effectively. This makes RoBERTa an appealing choice for predicting a diverse range of molecular properties, including solubility, reactivity, stability, and toxicity. Its comprehensive understanding of molecular data is attributed to its optimized training and advanced attention mechanisms, establishing it as a potent tool in molecular property prediction tasks.

The transformer-based network may be used to encode the SMILE string into a dense and expressive feature representation. In particular, the feature representation derived from the transformer-based network, encodes the sequential information present in the SMILES string. It captures the spatial arrangements, bond types, and other relationships between atoms as represented by the characters in the SMILES string. This feature representation focuses on understanding the sequential order and dependencies within the molecule.

The linear layer module 206 is a simple layer, but it is important because it allows the two modules (such as, the GNN module 202 and the transformer-based network module 204) to synergistically fuse their outputs. This means that the linear layer module 206 allows the two modules to work together to produce a more accurate prediction of the properties of a molecule

The linear layer module 206 may be implemented using a variety of different methods:

Simple matrix multiplication operation: The matrix multiplication operation takes the outputs of the two modules as input and outputs a single vector that represents the combined output of the two modules.

Weighted sum: This method simply takes the outputs of the two modules and sums them together, with each output weighted by a coefficient. This is a simple and efficient method, but it may not be as accurate as other methods.

Attention: This method uses a technique called attention to combine the outputs of the two modules. Attention allows the linear layer module to learn how to weight the outputs of the two modules in a way that produces a more accurate prediction of the properties of a molecule.

Neural network: This method uses a neural network to combine the outputs of the two modules. The neural network may be trained to learn how to combine the outputs of the two modules in a way that produces a more accurate prediction of the properties of a molecule

The linear layer module 206 may be used to combine the outputs of the GNN module 202 and the transformer-based network module 204. The linear layer module 206 takes a unified input feature generated by concatenating the features from both the GNN and Transformer Modules, and outputs a single vector.

Further, the predictor network module 208 may employ a predictor network to make predictions about the properties of a molecule based on the synergistic combined feature representation. The predictor network is trained to learn the relationship between the synergistic combined feature representation of the molecule and its properties. The predictor network may be trained using either a regression algorithm or a classification algorithm.

Synergy is a phenomenon in which the interaction of two or more substances produces an effect that is greater than the sum of their individual effects. This interaction leads to an enhancement or amplification of their overall impact. For instance, let's consider two substances labeled as A and B, each having distinct effects denoted by variables X and Y, respectively. When these substances interact, their combined effect is represented as Z. The presence of synergy between A and B can be mathematically expressed as Z>X+Y. In simpler terms, this equation signifies that the combined effect (Z) is greater than what would be expected by adding the individual effects (X+Y) together. This observation indicates the occurrence of a synergistic interaction.

The regression algorithms are used to predict a continuous value, such as the solubility of a molecule. This may be done by fitting a line or curve to the data points in the training set. The line or curve represents the relationship between the predictor variables and the target variable. Once the line or curve is fitted, it may be used to predict the value of the target variable for new data points.

Additionally, the classification algorithms are used to predict a categorical value, such as the toxicity of a molecule. They do this by creating a model that assigns each data point to a category. The model is created by training the algorithm on a set of data points that have already been classified. Once the model is trained, it may be used to predict the category of new data points.

Loss function may be used to train both regression algorithms and classification algorithms. In case of the regression algorithm, the loss function is typically a measure of the squared error between the predicted and ground truth values. In case of the classification algorithm, the loss function is typically a measure of the misclassification rate. The loss function is a measure of the error between the predicted properties of a molecule and the ground truth properties of the molecule. The goal of the predictor network is to minimize the loss function.

The loss function may be minimized using a technique called backpropagation. The backpropagation is a gradient-based optimization algorithm that iteratively adjust weights of the predictor network in order to minimize the loss function. The backpropagation works by propagating the error backwards through the neural network. The error is propagated from the output layer of the neural network to the input layer. At each layer, the error is used to adjust the weights of the layer in a way that reduces the error. The backpropagation process is repeated until the loss function is minimized.

The adjusted weights of the predictor network are then used to make predictions about the properties of new molecules. The predictions of the predictor network are called predictions. The predictions are made by passing the synergistic combined feature representation of a new molecule to the predictor network. The predictor network then outputs a prediction for the properties of the new molecule.

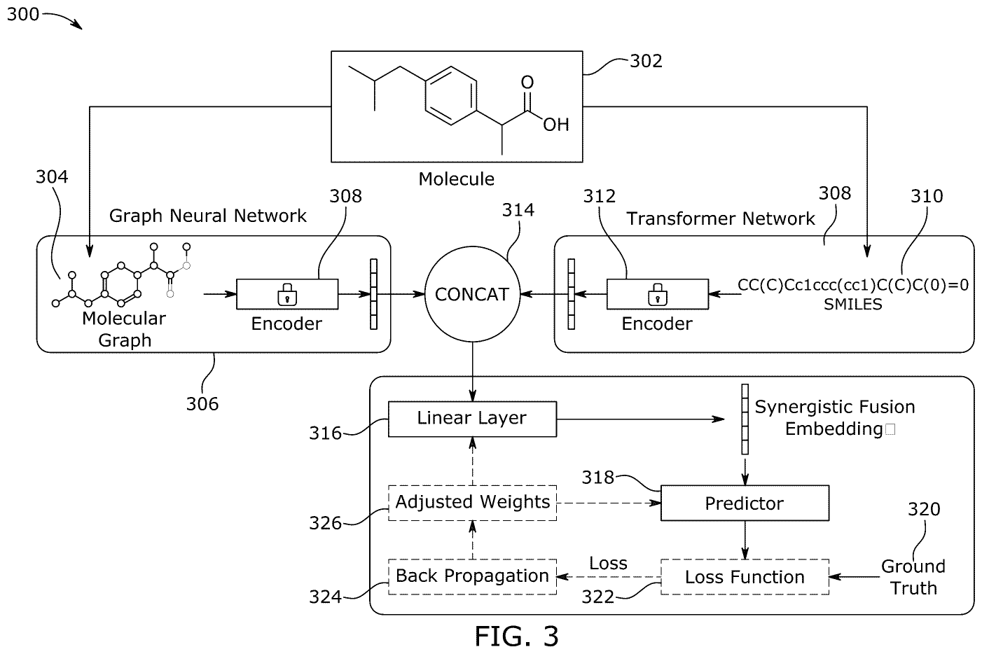

FIG. 3 illustrates a functional block diagram 300 for predicting molecular properties, in accordance with an example embodiment. The process starts with representing a singular molecule 302 as a graph (e.g., molecular graph 304) and a string (e.g., SMILES string 310). It should be noted that the graph representation is a representation of the molecule 302 as a network of nodes representing atoms and edges representing bonds. The string representation of the molecule 302 is a sequence of characters that represents the atoms and bonds in the molecule 302.

The next step is to encode the molecular graph 304 and SMILES string 310 representations of the molecule 302 into feature representations. In particular, the graph representation is encoded 308 using a GNN (such as, GIN), and the string representation is encoded 312 using a transformer-based network (such as, BERT, BART, etc.).

The feature representations obtained from the GNN, and transformer-based network are then concatenated 314. This concatenation step merges the distinct structure captured by the GNN and the transformer-based network. The concatenated feature representations effectively combine the distinct perspectives, forming a unified foundation for a subsequent fusion process.

The synergistic combined feature representation is created by fusing the concatenated feature representations through a linear layer 316. This linear layer 316 serves as a mechanism for combining and transforming the concatenated features. The linear layer 316 applies a weighted sum or other mathematical operations to adaptively blend the GNN and transformer-based features. By assigning suitable weights, the linear layer 316 optimally integrates the two sources of information, effectively achieving synergistic combination.

The fusion process facilitated by the linear layer 316 ensures that the molecular representation benefits from both the structure-based and sequential attributes. This enhanced representation captures a global structure of the molecule as well as the intricate characteristics of individual atoms, resulting in a more comprehensive and informative feature representation.

Further, the synergistic combined feature representation is fed into a predictor network 318. This predictor network 318 is a neural network specially designed and trained to predict molecular properties based on feature representations. It utilizes the encoded information to make informed predictions about various characteristics or behaviours of the molecule 302.

The output of predictor network 318 is a prediction regarding the molecular properties of the given molecule 302. The prediction may also be used to make decisions about the molecule, such as whether to synthesize the molecule 302 or to test the molecule 302 for toxicity. This prediction may provide valuable information related to the behaviour and attributes of the molecule 302, aiding in scientific understanding and decision-making processes.

The prediction of the predictor network 318 may be compared against a known ground truth 320, which represents the actual molecular properties of the molecule 302. This comparison may be facilitated by a loss function 322, which quantifies the discrepancy between the predicted properties and the ground truth 320. The loss function 322 measures an accuracy of the predictor network's predictions. To refine the predictor network performance, a process called back propagation 324 is employed. The back propagation 324 computes the gradients of the loss with respect to prediction network parameters, allowing the network to understand how to adjust its weights and biases to minimize the loss. The adjusted weights 326 acquired through the back propagation 324, optimize both the parameters of the predictor network 318 and the linear network. These optimized weights refine the representations and interactions of the prediction network, leading to improved accuracy in predictions.

For better understanding of the above-described process, consider a scenario of a molecule with known ground truth properties, such as solubility, toxicity, reactivity, and biological activity. Subsequently, assume that a trained predictor network provides the following predictions for the molecule: medium solubility, low toxicity, high reactivity, and inactive biological activity.

To assess the accuracy of these predictions, a suitable loss function, such as the Mean Squared Error (MSE) may be employed. The MSE calculates the average squared difference between the predicted values and the corresponding ground truth values for all the properties. By computing the MSE, a quantitative measure is obtained for how well the predictor network's predictions align with the actual ground truth values.

Assuming that the computed MSE for this case is lower, a lower MSE suggests that the predictor network's predictions are closer to the ground truth values, indicating a higher degree of accuracy. In this specific instance, the lower MSE implies that the predictor network's predictions exhibit a moderate level of accuracy.

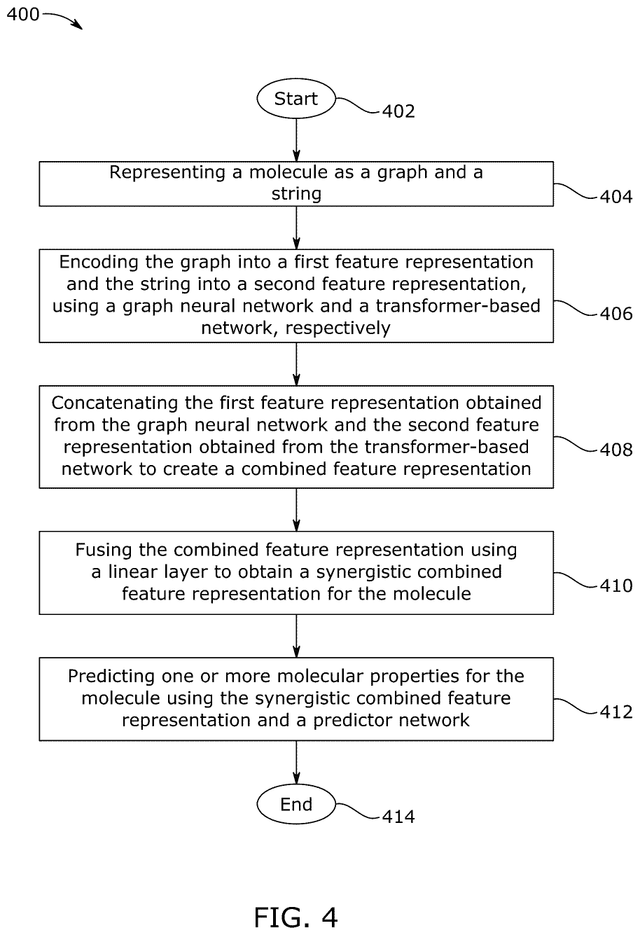

FIG. 4 illustrates a flow diagram of a method 400 for predicting molecular properties, in accordance with an example embodiment. It will be understood that each block of the flow diagram of the method 300 may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory 106 of the prediction device 102, employing an embodiment of the present disclosure and executed by a processor 108. As will be appreciated, any such computer program instructions may be loaded onto a computer or other programmable apparatus (for example, hardware) to produce a machine, such that the resulting computer or other programmable apparatus implements the functions specified in the flow diagram blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flow diagram blocks.

Accordingly, blocks of the flow diagram support combinations of means for performing the specified functions and combinations of operations for performing the specified functions for performing the specified functions. It will also be understood that one or more blocks of the flow diagram, and combinations of blocks in the flow diagram, may be implemented by special purpose hardware-based computer systems which perform the specified functions, or combinations of special purpose hardware and computer instructions.

The method 400 illustrated by the flow diagram of FIG. 4 for predicting molecular properties may start at step 402. Further, the method 400 may include representing a molecule as a graph and a string, at step 404. The graph representation may include nodes representing atoms and edges representing bonds. This graph structure provides a comprehensive understanding of the molecular connectivity and spatial arrangement of atoms and bonds.

Additionally, the string representation corresponds to a Simplified Molecular Input Line Entry System (SMILES). The SMILES string is a textual representation of the molecule, where characters and symbols encode the atom and bond information in a specific sequence, reflecting the molecular structure in a compact and standardized format.

The method 400, at step 406, may include encoding the graph into a first feature representation and the string into a second feature representation, using a graph neural network and a transformer-based network, respectively. The GNN is capable of learning and extracting meaningful features from the graph's node and edge attributes, capturing atom-level and bond-level information, as well as considering the structural relationships between atoms.

Simultaneously, the SMILES string that is encoded into a second feature representation using the transformer-based network, such as BERT, BART, etc., employs attention mechanisms to understand the sequential relationships and dependencies within the SMILES string, capturing important contextual information about the molecular structure.

At step 408, the method 400 may include, concatenating the first feature representation obtained from the graph neural network and the second feature representation obtained from the transformer-based network to create a combined feature representation.

The concatenation operation merges the two sets of feature vectors, resulting in a unified and elongated feature representation that contains information from both the graph and the sequential representation of the molecule.

At step 410, the method 400 may include, fusing the combined feature representation using a linear layer to obtain a synergistic combined feature representation for the molecule. It should be noted that the synergistic combined feature representation may capture global structure of molecules and characteristics of individual atoms to accurately predict the one or more molecular properties.

In a more elaborative way, the combined feature representation is passed through the linear layer. The linear layer applies a weighted sum or other mathematical operations to adaptively blend the concatenated features. By assigning suitable weights, the linear layer optimally integrates the graph and transformer-based features, effectively achieving a harmonious and synergistic combination.

The resulting synergistic combined feature representation captures both the global structure of the molecule and the intricate characteristics of individual atoms. This enriched representation serves as a detailed molecular feature, enhancing the accuracy and effectiveness of predicting various molecular properties.

At step 412, the method 400 may include, predicting one or more molecular properties for the molecule using the synergistic combined feature representation and a predictor network.

In this final step, the synergistic combined feature representation is utilized as input to a predictor network. The predictor network is a trained neural network specifically designed for molecular property prediction tasks. The predictor network utilizes the synergistic combined feature representation to make informed predictions about various characteristics or properties of the molecule, such as solubility, toxicity, reactivity, or biological activity. The predictions provide valuable information related to the behavior and properties of the molecule, enabling informed decision-making in various scientific and industrial applications. Further, the method 400 terminated at step 414.

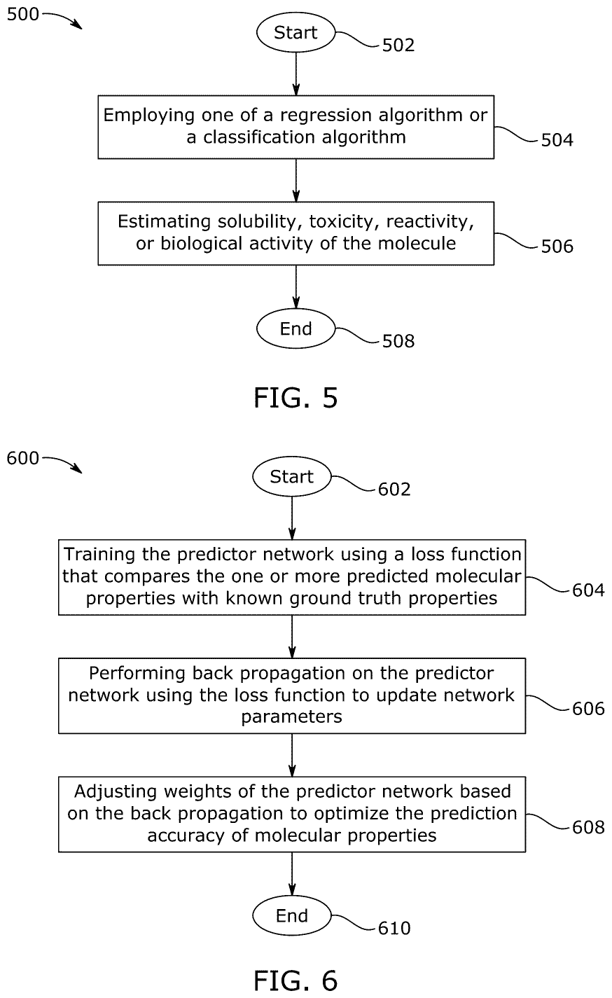

FIG. 5 illustrates a flow diagram of a method 500 for predicting molecular properties, in accordance with an example embodiment. It will be understood that each block of the flow diagram of the method 500 may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory 106 of the prediction device 102, employing an embodiment of the present disclosure and executed by a processor 108. As will be appreciated, any such computer program instructions may be loaded onto a computer or other programmable apparatus (for example, hardware) to produce a machine, such that the resulting computer or other programmable apparatus implements the functions specified in the flow diagram blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flow diagram blocks.

Accordingly, blocks of the flow diagram support combinations of means for performing the specified functions and combinations of operations for performing the specified functions for performing the specified functions. It will also be understood that one or more blocks of the flow diagram, and combinations of blocks in the flow diagram, may be implemented by special purpose hardware-based computer systems which perform the specified functions, or combinations of special purpose hardware and computer instructions.

FIG. 5 is explained in conjunction with elements from FIGS. 1, 2, 3, and 4. As mentioned in FIG. 4, once the synergistic combined feature representation for the molecule is obtained, the predictor network may be utilized to predict the one or more molecular properties. The present FIG. 5 illustrates the method 500 for predicting the one or more molecular properties using the predictor network.

At step 502, the method 500 is initiated. The method 500 at step 504, may include employing, by the predictor network, one of a regression algorithm or a classification algorithm for predicting molecular properties. The regression algorithm is used when the target variable (molecular property) is continuous, and the goal is to predict a numerical value, such as predicting the solubility value of a molecule on a continuous scale.

On the other hand, the classification algorithm is used when the target variable is categorical, and the goal is to predict which category a molecule belongs to, such as predicting if a molecule is toxic or non-toxic. The utilization of classification model and regression model for predicating toxicity and solubility of the molecule is depicted via Table 700 and Table 800.

Upon employing one of the regression algorithm or the classification algorithm, the method 500, at step 506 may further include estimating solubility, toxicity, reactivity, or biological activity of the molecule. The method 500 ends at step 508.

Estimating solubility, toxicity, reactivity, or biological activity of the molecule refers to the process of using the predictor network to make predictions about these specific properties based on the synergistic combined feature representation obtained earlier in the method. Here is the explanation of each of these terms in context of the molecular property prediction:

Solubility: Solubility refers to the ability of a substance (in this case, a molecule) to dissolve in a solvent and form a homogeneous solution. It is a crucial property in drug development, as it affects the bioavailability and effectiveness of drugs. By estimating the solubility of a molecule, researchers can determine how well the molecule will dissolve in various solvents or biological fluids, which is essential for drug formulation and delivery.

Toxicity: Toxicity is a measure of the harmful effects that a molecule may have on living organisms. Estimating the toxicity of a molecule is vital in drug discovery and safety assessment. By predicting the toxicity, researchers can identify potentially harmful molecules early in the drug development process, which helps in avoiding adverse effects on patients or the environment.

Reactivity: Reactivity refers to the tendency of a molecule to undergo chemical reactions. It is crucial in understanding how a molecule will interact with other molecules or substances in a chemical reaction. Predicting the reactivity of a molecule is essential in designing efficient chemical processes and understanding its potential for chemical transformations.

Biological Activity: Biological activity refers to the specific effects that a molecule may have on biological systems, such as binding to a target protein or modulating a biological pathway. Estimating the biological activity of a molecule is critical in drug discovery, as it helps identify potential drug candidates that can interact with specific biological targets and produce the desired therapeutic effects.

If the employed algorithm is regression, the predictor network may predict the solubility of the molecule as a continuous value, such as “0.45 grams per litre”. Moreover, if the employed algorithm is classification, the predictor network may predict the toxicity of the molecule as a categorical label, such as “Toxic” or “Non-toxic.”

The estimation of these molecular properties provides valuable information about the behavior and characteristics of the molecule, which can be crucial for various scientific and industrial applications in drug discovery, material science, environmental studies.

FIG. 6 illustrates a flow diagram of a method 600 for training prediction network, in accordance with an example embodiment. It will be understood that each block of the flow diagram of the method 600 may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory 106 of the prediction device 102, employing an embodiment of the present disclosure and executed by a processor 108. As will be appreciated, any such computer program instructions may be loaded onto a computer or other programmable apparatus (for example, hardware) to produce a machine, such that the resulting computer or other programmable apparatus implements the functions specified in the flow diagram blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flow diagram blocks.

Accordingly, blocks of the flow diagram support combinations of means for performing the specified functions and combinations of operations for performing the specified functions for performing the specified functions. It will also be understood that one or more blocks of the flow diagram, and combinations of blocks in the flow diagram, may be implemented by special purpose hardware-based computer systems which perform the specified functions, or combinations of special purpose hardware and computer instructions.

FIG. 6 is explained in conjunction with elements from FIGS. 1, 2, 3, 4, and 5. As discussed in reference to FIG. 4, the one or more molecular properties may be predicated for the molecule using the synergistic combined feature representation and the predictor network. Therefore, the present FIG. 6 illustrates the method 600 for training the predictor network to accurately predict the molecular properties.

At step 602, the method 600 is started. At step 604, the method 600 may include training the predictor network using a loss function that compares the one or more predicted molecular properties with known ground truth properties. The loss function quantifies the discrepancy between the predicted values generated by the predictor network and the actual ground truth values of the molecular properties. The ground truth values represent the true or known values of the molecular properties, typically obtained through experimental measurements or expert annotations.

During the training process, the predictor network iteratively makes predictions for a set of training data, and the loss function evaluates how well these predictions match the corresponding ground truth values. The goal of training is to minimize the loss, which means the predictor network should produce predictions that are as close as possible to the ground truth values. This process helps the predictor network learn from the data and adjust its internal parameters (weights and biases) to improve its predictive performance.

The method 600 may further include performing back propagation on the predictor network using the loss function to update network parameters, at step 606. Back propagation is a key algorithm used in training neural networks, including the predictor network in this method. After evaluating the loss function, back propagation calculates the gradients of the loss with respect to the network's parameters. These gradients represent the direction and magnitude of the change needed in the network's parameters to reduce the loss.

Once the gradients are computed, the back propagation algorithm applies the gradients to update the network's parameters through a process called gradient descent. Gradient descent involves adjusting the weights and biases of the predictor network in the opposite direction of the gradients, thereby nudging the network towards the configuration that minimizes the loss.

At step 608, the method 600 may include adjusting weights of the predictor network based on the back propagation to optimize the prediction accuracy of molecular properties. In this step, the weights of both the predictor network and the linear network are updated based on the gradients derived from back propagation. By adjusting the weights, the predictor network fine-tunes its internal representations and interactions between the feature representations to optimize its prediction accuracy of molecular properties.

The process of adjusting the weights continues iteratively during the training phase, with the predictor network making incremental improvements in its predictive capabilities. As the training progresses, the network's performance improves, and it becomes better at predicting molecular properties based on the synergistic combined feature representation obtained from the previous steps.

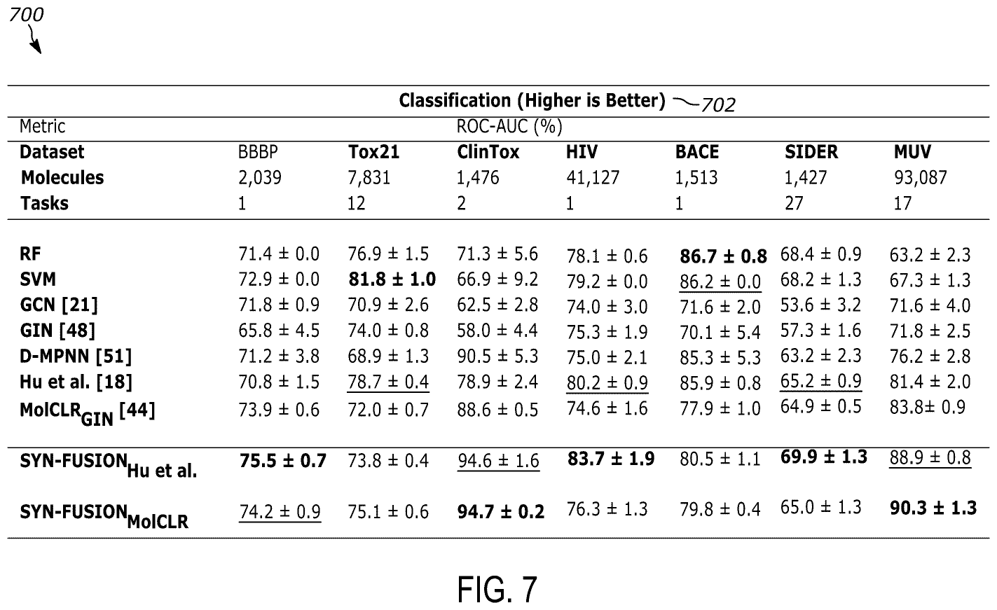

FIG. 7 shows an exemplary table 700 depicting comparison of a performance of different models for predicting a toxicity of molecules, in accordance with an example embodiment. The table 700 represent a ROC-AUC (Receiver Operating Characteristic—Area Under the Curve) scores of the different models on seven different datasets. ROC-AUC is a metric that is used to measure the performance of a classification model. A higher ROC-AUC score indicates a better is the classification model at accurate predication.

The term “Classification (higher is better) 702” at the top of the table 700 indicates that the ROC-AUC scores are a measure of the performance of the models for classification tasks. In classification tasks, the goal is to predict the category of a data point. In this case, the goal is to predict whether a molecule is toxic or not.

A higher ROC-AUC score indicates that the model is better at making accurate predictions. This is because a higher ROC-AUC score means that the model is better at distinguishing between the two categories (toxic and non-toxic).

The present FIG. 7 shows that the proposed SYN-FUSION (such as synergically fusion of GNN and transformer based network) has the highest ROC-AUC scores on 5 out of 7 datasets such as, BBBP, ClinTox, HIV, SIDER, and MUV. The relative improvement of SYN-FUSION over Hu et. al and MolCLR is shown in the present table 700. For example, on the BBBP dataset, SYN-FUSION has a relative improvement of 6.63% over Hu et. al and 0.4% over MolCLR. Further, SYN-FUSION has a relative improvement of (6.63, 0.4) % on BBBP, (−6.2, 4.3) % on Tox21, (19.89, 6.88) % on ClinTox, (4.36, 2.27) % on HIV, (−6.2, 2.4) % on BACE, (7.2, 0.15) % on SIDER, (9.21, 7.75) % on MUV datasets when comparing against its non-fusion counterparts Hu et. al and MolCLR approaches respectively.

The study also found that SYN-FUSION outperformed the state-of-the-art model on the ClinTox and SIDER datasets. The state-of-the-art model is the model that was previously considered to be the best model for predicting molecular properties. This suggests that SYN-FUSION is a better performing model for predicting the toxicity of molecules than the other models that were evaluated in the study.

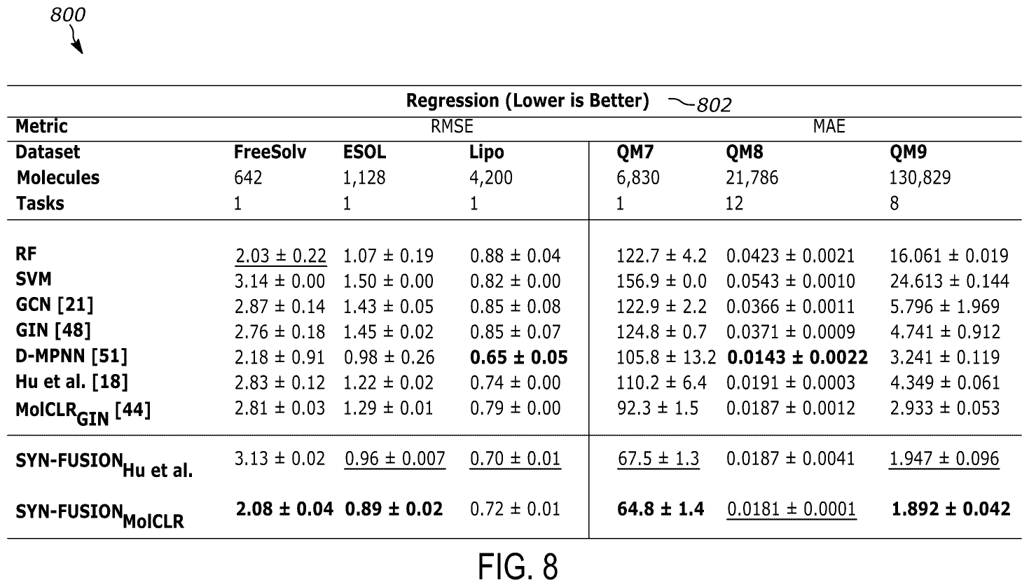

FIG. 8 shows an exemplary table 800 depicting a comparison of performance of different models for predicting a solubility of molecules, in accordance with an example embodiment. In particular, the present table 800 shows results of a study that compared the performance of different models for predicting the solubility of molecules. The table 800 represents a RMSE (Root Mean Square Error) scores of the different models on 6 different datasets. The RMSE is a metric that is used to measure the performance of a regression model. A lower RMSE score indicates that the regression model better at making accurate predictions.

The term “Regression (lower is better) 802” at the top of the table 800 indicates that the RMSE scores are a measure of the performance of the models for regression tasks. In regression tasks, the goal is to predict a continuous value. In this case, the goal is to predict the solubility of the molecule.

The study shows that SYN-FUSION has the lowest RMSE scores on 4 out of 6 datasets namely FreeSolv, ESOL, QM7, and QM9. These datasets encompass a diverse range of molecular properties, thereby providing a comprehensive evaluation of the fusion approach's capabilities. SYN-FUSION has a relative improvement of (−10.6, 25.97) % on FreeSolv, (21.3, 31) % on ESOL, (5.4, 8.86) % on Lipo, (38.74, 29.79) % on QM7, (2.09, 3.2) % on QM8, (55.23, 35.49) % on QM9 datasets when comparing on Hu et. al and MolCLR approaches respectively. Notably, the SYN-FUSION model demonstrated significant improvements over the previous best, achieving a remarkable 9.1% improvement on the ESOL dataset. This suggests that SYN-FUSION is a better performing model for predicting the solubility of molecules than the other models that were evaluated in the study.

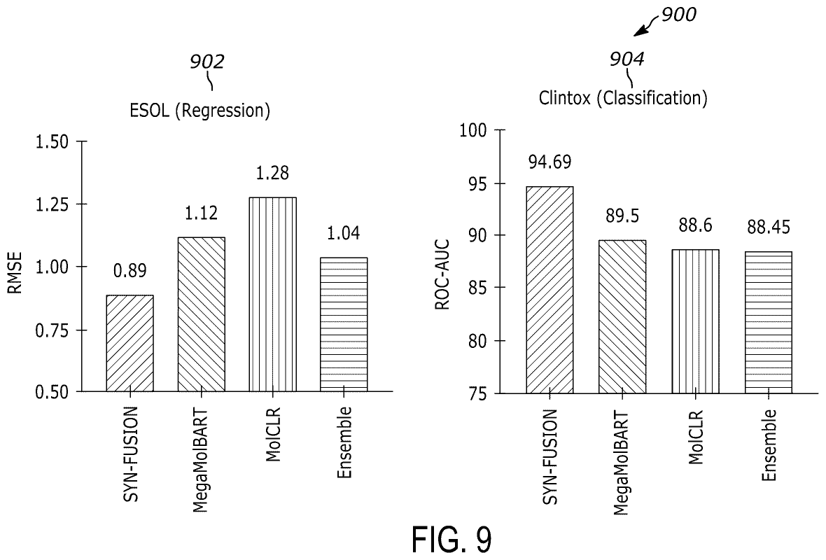

FIG. 9 shows an exemplary graphical representation 900 depicting comparison of a performance of different models for predicting a solubility and toxicity of molecules, in accordance with an example embodiment. In particular, the present FIG. 9 shows results of a study that compared the performance of different models for predicting the solubility represented by graph EOS (regression) 902 and toxicity represented by graph ClinTox (classification) 904 of molecules.

In order to experimentally verify the impact of synergy, a comparative analysis is performed between the combined effect (represented by SYN-FUSION) and the individual models (MolCLR and MegaMolBART), as well as their (sum) ensemble, on both classification and regression tasks. In the ensemble approach, the predictions generated by each individual model is handled differently depending on the task at hand.

For classification, if both models provided identical predictions, the prediction is retained as is. However, in cases where the models offered differing predictions, the prediction with higher confidence is considered. For regression, the average of the two individual model predictions is computed. In the absence of SYN-FUSION, the AUC % drops from 94.69% by 5.19%-6.24% on ClinTox, and the RMSE increases from 0.89 by 0.15-0.39 on ESOL. This demonstrates the synergy effect—the combined effect achieved through fusion is greater than the individual models and their ensemble.

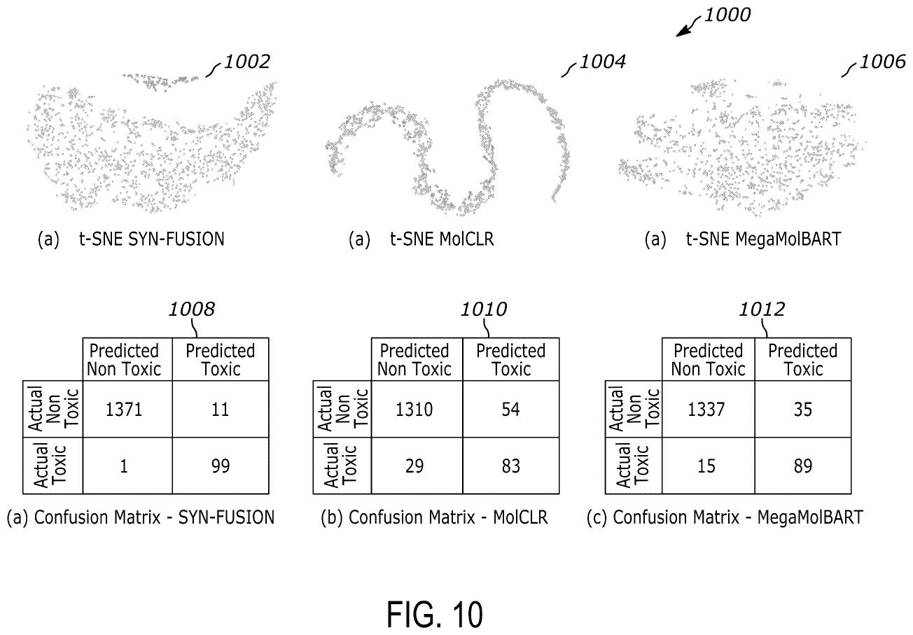

FIG. 10 shows a comparison of latent space representations 1000 of SYN-FUSION, MolCLR, and MegaMolBAR, in accordance with an example embodiment. The present FIG. 10 describes a study that is conducted to compare the latent space representations 1000 of the SYN-FUSION model and its individual components (such as, MolCLR and MegaMolBART) on the ClinTox classification dataset. The latent space representation is a way of mapping molecules to a lower-dimensional space where they may be more easily visualized and analyzed.

The study found that the latent space representations of SYN-FUSION have a clear separation between the two classes of molecules, toxic and non-toxic. This means that the toxic molecules are clustered together in one region of the latent space, while the non-toxic molecules are clustered together in another region. This separation is not as clear in the latent space representations of MolCLR and MegaMolBART.

The study also found that SYN-FUSION made fewer incorrect predictions in distinguishing between toxic and non-toxic molecules than MolCLR and MegaMolBART. This is consistent with the finding that the latent space representations of SYN-FUSION have a clearer separation between the two classes of molecules.

The t-SNE plots were generated to compare the latent space representations of the proposed SYN-FUSION model and its individual components (MolCLR and MegaMolBART) on the ClinTox classification dataset, providing qualitative visualization for comparison as shown by present FIG. 10, t-SNE SYN-FUSION 1002, t-SNE MolCLR 1004, and t-SNE MegaMolBART 1006 displaying the t-SNE plots of the embeddings derived from SYN-FUSION, MolCLR, and MegaMolBART, respectively. The t-SNE plot is a technique that may be used to visualize high-dimensional data in a lower-dimensional space. The red and blue points represent the projection of toxic and non-toxic molecule samples respectively. The t-SNE SYN-FUSION 1002 has a clear separation between the two classes, as the toxic samples cluster together at the top while the non-toxic molecules appear at the bottom. This observation indicates the successful learning and encoding of discriminative features pertaining to toxic and non-toxic molecules by SYN-FUSION, and the model's ability to make accurate predictions regarding the toxicity of new molecules. In contrast,

The t-SNE MolCLR 1004 suggests that the latent representations of toxic and non-toxic molecules in MolCLR are intermingled instead of having a separation. This finding implies that MolCLR may face difficulties in accurately classifying molecules as toxic or non-toxic. MegaMolBART (t-SNE MegaMolBART 1006) exhibits improved discrimination between toxic and non-toxic molecules, although there are still scattered instances of toxic molecules among the non-toxic ones, and the level of separation is not as pronounced as in SYN-FUSION.

The confusion matrices obtained on evaluation of approximately 1476 molecule samples from ClinTox is represented by confusion matrix—SYN-FUSION 1008, confusion matrix—MolCLR 1010, and confusion matrix—MegaMolBART 1012 show the results of the study. The confusion matrix is a table that is used to visualize the performance of a classification model. The rows of the confusion matrix represent the actual classes of the molecules, while the columns represent the predicted classes. The darker the cell, the more molecules in that class were predicted to be in the other class.

The confusion matrices show that SYN-FUSION made fewer incorrect predictions than MolCLR and MegaMolBART. This is consistent with the finding that the latent space representations of SYN-FUSION have a clearer separation between the two classes of molecules.

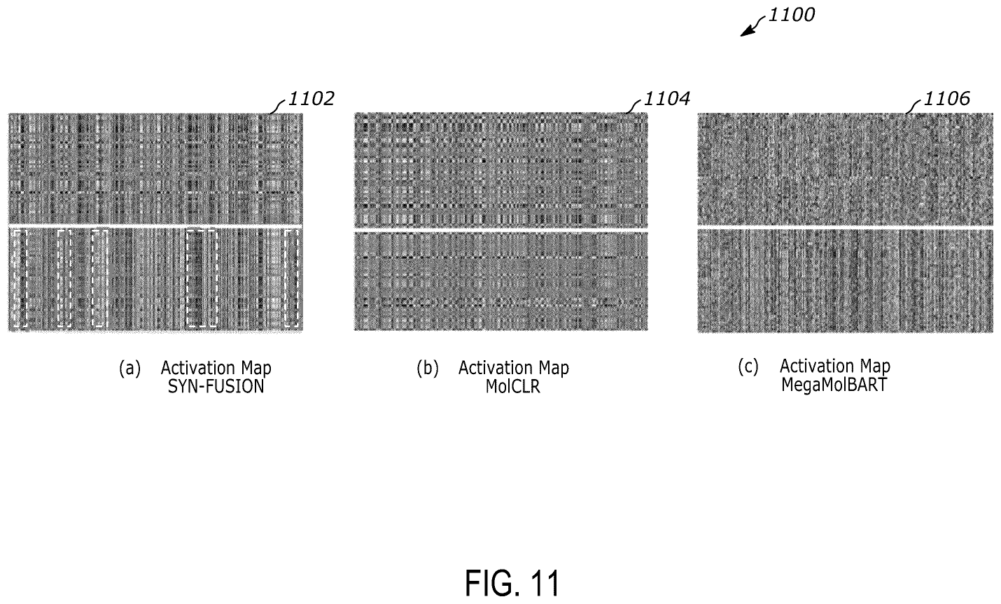

FIG. 11 illustrates exemplary Activation Maps 1100 of different models, in accordance with an example embodiment. The activation maps 1100 are a visualization of the attention weights that the models assign to different features of the molecules. The Activation maps 1100 help to identify similar patterns across samples belonging to the same class and enable the model to distinguish between classes, leading to effective decision-making.

To observe any learned patterns between toxic and non-toxic molecules, approximately 100 samples from ClinTox dataset were considered in equal proportions (50 toxic/50 non-toxic), and 1-D activation maps were generated using the last layer of SYN-FUSION, MolCLR, and MegaMolBART models. The present FIG. 11 showcases the stacked 1-D activation maps of SYN-FUSION 1102, revealing distinct and clear activation patterns across samples in both classes, indicating that the model has learned to focus on relevant features for effective classification.

In contrast, the activation maps of MolCLR 1104 lack well-defined patterns, and it is difficult to differentiate the toxic from the non-toxic class. Activation maps generated by MegaMolBART 1106 demonstrate intermediate characteristics between the two models, revealing a few discernible patterns that are slightly more noticeable compared to MolCLR but not as prominent as SYNFUSION.

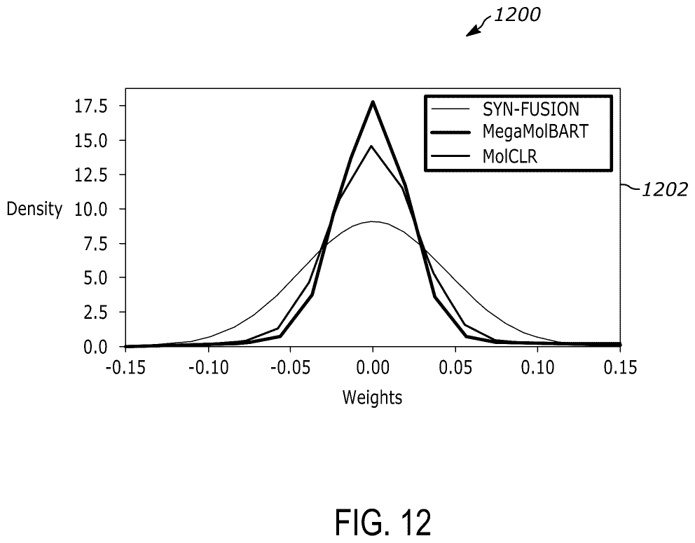

FIG. 12 illustrates exemplary weight histograms 1200 of different models, in accordance with an example embodiment. In particular, a weight histogram 1202 represents density of SYN-FUSION, MolCLR, and MegaMolBART with respect to weight. A weight histogram shows the distribution of weights within a model, providing insights into the range and frequency of different weight values.

Small weights (values close to 0.0) tend to yield sharper minimizers and exhibit greater sensitivity to perturbations. Conversely, weight distribution with uniform variance (both positive and negative values) leads to flatter minima and contributes to better generalization. In light of this finding, the weight distributions of the final layer of SYN-FUSION, MolCLR, and MegaMolBART were investigated after completion of training, and the histogram of weights is presented.

From the present FIG. 12, it is observed that SYN-FUSION produces higher magnitude (both range and density) weights compared to MolCLR and MegaMolBART, indicating that fusion improves generalization and helps in easier and faster optimization. The impact of this phenomenon may be observed in the loss interpolation where SYN-FUSION demonstrates a favorable initialization denoted by a lower initial loss value, undergoes a rapid minimization of the loss ending with a significantly lower final loss value.

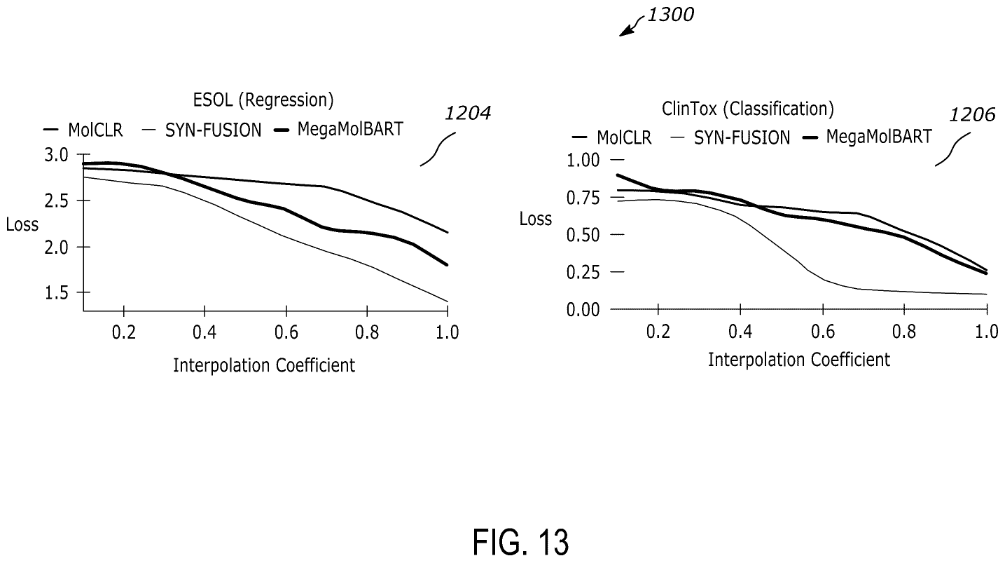

FIG. 13 illustrates exemplary loss interpolation plots 1300 of different models, in accordance with an example embodiment. Loss interpolation plots (as depicted by ESOL (regression) 1304, and ClinTox (classification) 1306) offer a concise visualization of the transition between different loss values, providing valuable insights into the behavior and convergence of optimization over the course of training. Notably, the presence of Monotonic Linear Interpolation in a model's loss trajectory signifies that the optimization of tasks is relatively easier. Notable differences are observed in the loss trajectories of the models under comparison, as shown in present FIG. 13 Specifically, the loss curve of SYN-FUSION displays a remarkably high level of monotonicity, suggesting a smoother and more consistent optimization process, in contrast to the loss curves of MolCLR and MegaMolBART. Moreover, SYN-FUSION has lower initial and final loss values compared to the other two models, providing additional evidence of easier and better optimization. These findings substantiate the effectiveness of synergistic fusion surpassing individual models in terms of optimization and convergence.

As will be appreciated by those skilled in the art, the techniques described in the various embodiments discussed above are not routine, or conventional, or well understood in the art. The techniques discussed above provide for predicting molecular properties with a novel approach that synergistically combines pre-trained features from the graph neural networks (GNNs) and transformer-based networks. This synergistic fusion enables the creation of a comprehensive molecular representation, capturing both the global structure of molecules and the fine-grained characteristics of individual atoms. The incorporation of the GNN facilitates the modeling of intricate connectivity patterns and spatial arrangements found in molecular graphs, while the transformer-based network excels in learning complex relationships and hidden dependencies across the entire molecule. The fusion of these pre-trained features enriches the molecular representation, leading to enhanced predictive accuracy for various molecular properties, such as solubility, toxicity, reactivity, and biological activity.

Further, the techniques provide various technical benefits such as improved prediction accuracy compared to conventional methods, greater understanding of molecular behavior, and potential applications in drug discovery, material design, and environmental analysis. Additionally, the techniques allow for more informed decision-making in synthesizing and testing molecules, optimizing research efforts, and accelerating advancements in computational chemistry.

Moreover, the utilization of machine learning techniques, including back propagation and loss functions, empowers the model to adaptively learn and optimize its parameters, further enhancing its predictive capabilities. The versatility of the proposed techniques enables their application in diverse molecular property prediction tasks, offering valuable insights for scientific and industrial purposes.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

It is to be understood that the above description is intended to be illustrative, and not restrictive. For example, the above-discussed embodiments may be used in combination with each other. Many other embodiments will be apparent to those of skill in the art upon reviewing the above description.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions, and improvements to the embodiments described above are possible. It is contemplated that these variations, modifications, additions, and improvements fall within the scope of the invention.

We claim:

1. A computer-implemented method for predicting molecular properties, comprising:
representing a molecule as a graph and a string;
encoding the graph into a first feature representation and the string into a second feature representation, using a graph neural network and a transformer-based network, respectively;
concatenating the first feature representation obtained from the graph neural network and the second feature representation obtained from the transformer-based network to create a combined feature representation;
fusing the combined feature representation using a linear layer to obtain a synergistic combined feature representation for the molecule; and
predicting one or more molecular properties for the molecule using the synergistic combined feature representation and a predictor network.

2. The computer-implemented method of claim 1, wherein the graph neural network include architectures selected from, but not limited to, a Graph Isomorphism Network (GIN), a Graph Convolutional Network (GCN), and a Graph Attention Networks (GAT), and wherein the transformer based network include architectures selected from, but not limited to, Bidirectional Encoder Representations from Transformers (BERT), Bidirectional Autoregressive Transformers (BART), and Robustly Optimized BERT Pretraining Approach (RoBERTa).

3. The computer-implemented method of claim 1, wherein the graph representation comprises nodes representing atoms and edges representing bonds.

4. The computer-implemented method of claim 1, wherein the string corresponds to one of a Simplified Molecular Input Line Entry System (SMILES), a SELF-referencIng Embedded Strings (SELFIES), or an International Chemical Identifier (InChi).

5. The computer-implemented method of claim 4, wherein the SMILES string, SELFIES string, or InChi are textual representations of the molecule.

6. The computer-implemented method of claim 1, wherein predicting the one or more molecular properties comprises estimating solubility, toxicity, reactivity, or biological activity of the molecule.

7. The computer-implemented method of claim 1, wherein the predictor network is a neural network that employ one of a regression algorithm or a classification algorithm for predicting molecular properties.

8. The computer-implemented method of claim 1, further comprising training the predictor network using a loss function that compares the one or more predicted molecular properties with known ground truth properties.

9. The computer-implemented method of claim 8, further comprising:
performing back propagation on the predictor network using the loss function to update network parameters; and
adjusting weights of the predictor network based on the back propagation to optimize the prediction accuracy of molecular properties.

10. The computer-implemented method of claim 1, wherein the synergistic combined feature representation captures global structure of molecules and characteristics of individual atoms to accurately predict the one or more molecular properties.

11. A computer system for predicting molecular properties, the computer system comprising: one or more computer processors, one or more computer readable memories, one or more computer readable storage devices, and program instructions stored on the one or more computer readable storage devices for execution by the one or more computer processors via the one or more computer readable memories, the program instructions comprising:
representing a molecule as a graph and a string;
encoding the graph into a first feature representation and the string into a second feature representation, using a graph neural network and a transformer-based network, respectively;
concatenating the first feature representation obtained from the graph neural network and the second feature representation obtained from the transformer-based network to create a combined feature representation;
fusing the combined feature representation using a linear layer to obtain a synergistic combined feature representation for the molecule; and
predicting one or more molecular properties for the molecule using the synergistic combined feature representation and a predictor network.

12. The computer system of claim 11, wherein the graph neural network include architectures selected from, but not limited to, a Graph Isomorphism Network (GIN), a Graph Convolutional Network (GCN), and a Graph Attention Networks (GAT), and wherein the transformer based network include architectures selected from, but not limited to, Bidirectional Encoder Representations from Transformers (BERT), Bidirectional Autoregressive Transformers (BART), and Robustly Optimized BERT Pretraining Approach (RoBERTa).

13. The computer system of claim 11, wherein the graph representation comprises nodes representing atoms and edges representing bonds.

14. The computer system of claim 11, wherein the string corresponds to a Simplified Molecular Input Line Entry System (SMILES), a SELF-referencIng Embedded Strings (SELFIES), or an International Chemical Identifier (InChi).

15. The computer system of claim 14, wherein the SMILES string and the SELFIES, or InChi are textual representations of the molecule.

16. The computer system of claim 11, wherein predicting the one or more molecular properties comprises estimating solubility, toxicity, reactivity, or biological activity of the molecule.

17. The computer system of claim 11, wherein the predictor network is a neural network that employ one of a regression algorithm or a classification algorithm for predicting molecular properties.

18. The computer system of claim 11, further comprising training the predictor network using a loss function that compares the one or more predicted molecular properties with known ground truth properties.

19. The computer system of claim 11, further comprising:
performing back propagation on the predictor network using the loss function to update network parameters; and
adjusting weights of the predictor network based on the back propagation to optimize the prediction accuracy of molecular properties.

20. A non-transitory computer-readable storage medium having stored thereon computer executable instruction which when executed by one or more processors, cause the one or more processors to carry out operations for hiding personal application user interface (UI) elements, the operations comprising perform the operations comprising:
representing a molecule as a graph and a string;
encoding the graph into a first feature representation and the string into a second feature representation, using a graph neural network and a transformer-based network, respectively;
concatenating the first feature representation obtained from the graph neural network and the second feature representation obtained from the transformer-based network to create a combined feature representation;
fusing the combined feature representation using a linear layer to obtain a synergistic combined feature representation for the molecule; and
predicting one or more molecular properties for the molecule using the synergistic combined feature representation.
